(19) United States
(12) Patent Application Publication
(10) Pub. No.: US 2026/0258041 A1
(43) Pub. Date: Sep. 3, 2026

(54) PROCESS FOR THE PRODUCTION OF HIGH PURITY MITRAGYNINE

(71) Applicant: LUCKY 4U EXIMS, Tirupati, Andhra Pradesh (IN)

(72) Inventors: Surya Pavan Kumar AVVA, Tirupati, Andhra Pradesh (IN); V.S.R. Suresh MAMIDIPALLI, Tirupati, Andhra Pradesh (IN); Lakshmipathi Raju DANDU, Hyderabad, Telangana (IN)

(21) Appl. No.: 18/995,804

(22) PCT Filed: Oct. 12, 2023
(86) PCT No.: PCT/IN2023/050930

§ 371 Date: Jan. 16, 2025

(30) Foreign Application Priority Data
Jul. 20, 2023 (IN) 202341048746

(51) Int. Cl. C07D 471/14

(52) U.S. Cl. CPC C07D 471/14

(57) ABSTRACT

The present invention discloses a novel process for the purification of Mitragynine. Particularly present invention discloses a process for the production of high purity Mitragynine for the pharmaceutical and medicinal preparations. The process includes purification of Mitragynine using column chromatography with mixture of polar aprotic & non-polar aprotic organic solvents in presence of an organic base.

### CROSS-REFERENCE TO RELATED PATENT APPLICATION(S)

The present application is a U.S. National Phase Entry of International PCT Application No. PCT/IN2023/050930 having an international filing date of Oct. 12, 2023, which claims the priority to, and benefit of Indian Patent Application number 202341048746 filed on Jul. 20, 2023. The disclosures of the above-identified applications are incorporated herein by reference.

### FIELD OF THE INVENTION

The present invention relates to a process for purification of Mitragynine. Particularly present invention relates to a process for the production of high purity Mitragynine for the pharmaceutical and medicinal preparations. More particularly, present invention relates to purification of Mitragynine using column chromatography.

### BACKGROUND OF THE INVENTION

Mitragynine is an alkaloid and the most abundant active alkaloid in the Southeast Asian plant Mitragyna speciosa, commonly known as kratom. It is found in the leaves of tree Mitragyna speciosa. Of all the other alkaloids present in plant Mitragyna speciosa, Mitragynine is considered to be largely responsible for its therapeutic activity.

Mitragynine was first isolated in 1921 and the chemical structure of Mitragynine was fully elucidated in 1964. The systematic (IUPAC) name of Mitragynine is methyl (E)-2-[(2S,3S,12bS)-3-ethyl-8-methoxy-1,2,3,4,6,7,12,12b-octahydroindolo[2,3-a]quinolizin-2-yl]-3-methoxyprop-2-enoate. The chemical structure of Mitragynine is represented as:

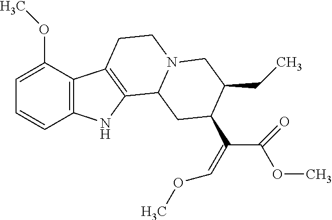 [SMILES: CC[C@@H]1CN2CCc3c([nH]c4cccc(OC)c34)C2C[C@@H]1/C(=C\OC)C(=O)OC]

Mitragynine possesses several pharmacological properties such as analgesic, anti-inflammatory, antipyretic, antidiarrheal, euphoric, antidepressant and anxiolytic effects.

Thiruventhan Karunakaran et. al. (2022) provides an insight review for the Chemical and Pharmacological properties of Mitragynine. The review reveals that Mitragynine has therapeutic potential for pain management as it has limited adverse effect compared to a classical opioid, morphine. Mitragynine is frequently regarded to behave like an opioid but possesses milder withdrawal symptoms. The use of this alkaloid as the source of an analgesic candidate has been proven through comprehensive preclinical and clinical studies.

Due to the increased interest in said alkaloid, an increasing amount of research are being conducted in isolating Mitragynine via various techniques. However, there are issues regarding the purity of said alkaloids isolated from Mitragyna speciosa plant due to the difficulty in separating and isolating isomeric alkaloid.

The difficulty has also been increased with respect to use of Mitragynine in pharmaceutical and medicinal preparations since a particular degree of purity is mandatory for said use. This has prompted researchers to develop such processes that ensures purity which is within the required guidelines as it is vital for pharmaceutical and medicinal preparations.

Laura Orio et. al. (2011) involves UAE, MAE, SFE-CO2 and classical methods for the extraction of Mitragyna speciosa leaves followed by purification by flash chromatography using petroleum ether and ethyl acetate as eluents. Purity of the obtained Mitragynine was estimated, by comparison of GC peak areas, as 94.17%.

Radhiahtul Raehan Mustafa et. al. (2020) involves extraction of the Mitragynine from M. speciosa leaves using a sequential solvent extraction method followed by purification using column chromatography to obtain Mitragynine having a purity of 98%.

Yong Sean Goh et. al. (2021) involves Accelerated Solvent Extraction (ASE) of Mitragyna speciosa leaves to obtain crude Mitragynine extract followed by purification using column packed with Sephadex Liphophilic LH-20 using methanol as the mobile phase. The obtained Mitragynine has 98% purity.

Purity standards relate directly to quality control in pharmaceutical production. The availability of impurities can completely alter a formulation and can produce something entirely different because chemicals react differently. For example, the wrong chemical components in a drug formulation can make a drug dangerous to human beings or animals. Therefore, chemical compounds with least possible impurities are always preferred in pharmaceutical production.

Considering the above fact, the present inventors have found simple and commercially significant process for obtaining high purity Mitragynine for pharmaceutical and medicinal preparations.

### OBJECTS OF THE INVENTION

The primary object of the present invention is to provide an improved process for production of high purity Mitragynine.

Another object of the present invention is to provide a process for purification of Mitragynine from crude Mitragynine compound by chromatographic technique, using mixture of non-polar aprotic solvent & polar aprotic solvents in presence of an organic base.

Another object of the present invention is to provide a facile, efficient, and cost-effective process for purification of crude Mitragynine which results in an optimum purity and stability of purified Mitragynine.

Another object of the present invention is to provide a process resulting in Mitragynine compound with purity above 99% by HPLC.

Yet another object of the present invention is to provide a high purity Mitragynine for pharmaceutical applications and medicinal preparations.

### SUMMARY OF THE INVENTION

Recognizing the prior arts limitations and need for improved process for purification of Mitragynine, in one aspect accordingly the present invention provides a process for the production of high purity Mitragynine comprising the steps of:
(a) loading crude Mitragynine compound (1) in a silica column;
(b) eluting the column with mixture of non-polar aprotic and polar aprotic organic solvents in presence of an organic base;
(c) collecting the main fraction obtained in step (b) and concentrating under reduced pressure to obtain crude Mitragynine compound (2);
(d) loading the crude Mitragynine compound (2) obtained in step (c) into the silica column;
(e) eluting the column with mixture of non-polar aprotic and polar aprotic organic solvents;
(f) collecting the main fraction obtained in step (e) and concentrating under reduced pressure to obtain the material Mitragynine;
(g) dissolving the obtained material in step (f) in an alcohol to obtain alcoholic solution of Mitragynine and passing through the molecular sieves for multiple times; and
(h) concentrating the filtered alcoholic solution of Mitragynine obtained in step (g) under the reduced pressure to obtain high purity Mitragynine.

The crude Mitragynine compound (1) employed in step (a) of the present process is 50-70% pure.

In the said process the non-polar aprotic organic solvent and polar aprotic organic solvent in steps (b) and (e) are present at a ratio 80:20, 70:30, 60:40, 50:50, 40:60, 30:70, or 20:80.

In step (b) of the present process the non-polar aprotic organic solvent and polar aprotic organic solvent are present at a ratio 60:40.

In step (e) of the present process the non-polar aprotic organic solvent and polar aprotic organic solvent are present at a ratio 80:20.

The non-polar aprotic organic solvent employed in steps (b) and (e) of the present process is selected from a group comprising of n-hexane, cyclohexane, pentane, heptane, carbon tetrachloride, benzene, toluene, or mixture thereof.

Preferably, the non-polar aprotic organic solvent employed in steps (b) and (e) is n-hexane.

The polar aprotic organic solvent employed in steps (b) and (e) of the present process is selected from a group comprising of acetone, acetonitrile, dimethyl sulfoxide, dimethylformamide, tetrahydrofuran, dichloromethane, ethyl acetate, or mixture thereof.

Preferably, the polar aprotic organic solvent employed in steps (b) and (e) is ethyl acetate.

The concentration of organic base employed in step (b) of the present process ranges between 1% to 5% (v/v).

Preferably, the concentration of organic base employed in step (b) is 2% (v/v).

The organic base employed in step (b) is selected from the group comprising of ammonia, amines, pyridine, aniline, or mixture thereof. Preferably, the organic base is amine selected from the group comprising of methylamine, dimethylamine, trimethylamine, ethylamine, diethylamine, benzylamine, butylamine, pentylamine, hexylamine, and octylamine. More preferably, the organic base employed in step (b) is triethylamine.

In step (b) of the said process the said column is eluted at a flow rate of 4-8 mL/min.

In step (e) of the said process the said column is eluted at a flow rate of 5-10 mL/min.

In the said process, the concentration under reduced pressure in step (c), (f) and (h) is performed using a rotary evaporator (Rotavapor).

The alcohol employed in step (g) of the said process is selected from methanol, ethanol, n-propanol, isopropanol, n-butanol, and tert-butanol.

Preferably, the alcohol employed in step (g) is methanol.

In step (g) of the said process the alcoholic solution of Mitragynine is passed through the molecular sieves for 2-3 times.

In some embodiment of the invention, there is provided a process for obtaining high purity Mitragynine, wherein said process comprises the steps of:
(a) loading crude Mitragynine compound (1) having purity 50-70% in a silica column;
(b) eluting the column with mixture of n-hexane and ethyl acetate in presence of triethylamine at a flow rate of 4-8 mL/min;
(c) collecting the main fraction obtained in step (b) and concentrating under reduced pressure to obtain crude Mitragynine compound (2);
(d) loading the crude Mitragynine compound (2) obtained in step (c) into the silica column;
(e) eluting the column with mixture of n-hexane and ethyl acetate at a flow rate of 5-10 mL/min;
(f) collecting the main fraction obtained in step (e) and concentrating under reduced pressure to obtain the material Mitragynine;
(g) dissolving the obtained material in step (f) in methanol to obtain methanolic solution of Mitragynine and passing through the molecular sieves for three times; and
(h) concentrating the methanolic solution of Mitragynine obtained in step (g) under the reduced pressure to obtain high purity Mitragynine.

In the said process the concentration under reduced pressure in steps (c), (f) and (h) is performed using a rotary evaporator (Rotavapor).

In the said process the ratio (v/v) of n-hexane to ethyl acetate is 80:20, 70:30, 60:40, 50:50, 40:60, 30:70, 20:80. Preferably the ratio of n-hexane to ethyl acetate, in step (b) is 60:40 (v/v) and in step (e) is 80:20 (v/v).

The concentration of triethylamine employed in step (b) of the said process ranges between 1% to 5% (v/v). Preferably, the concentration of triethylamine in step (b) is 2% (v/v).

The instant process results in high purity Mitragynine. In some embodiment the purity of obtained Mitragynine is above 99% by HPLC.

The obtained Mitragynine compound according to the present process is free of eluting solvent impurities.

The obtained high purity Mitragynine compound is storage stable at 2° C.-8° C.

In some embodiment of the present invention, high purity Mitragynine compound obtained according to present invention is suitable for the pharmaceutical preparations.

### BRIEF DESCRIPTION OF THE DRAWING

FIG. 1 depicts analytical data of obtained Mitragynine compound.

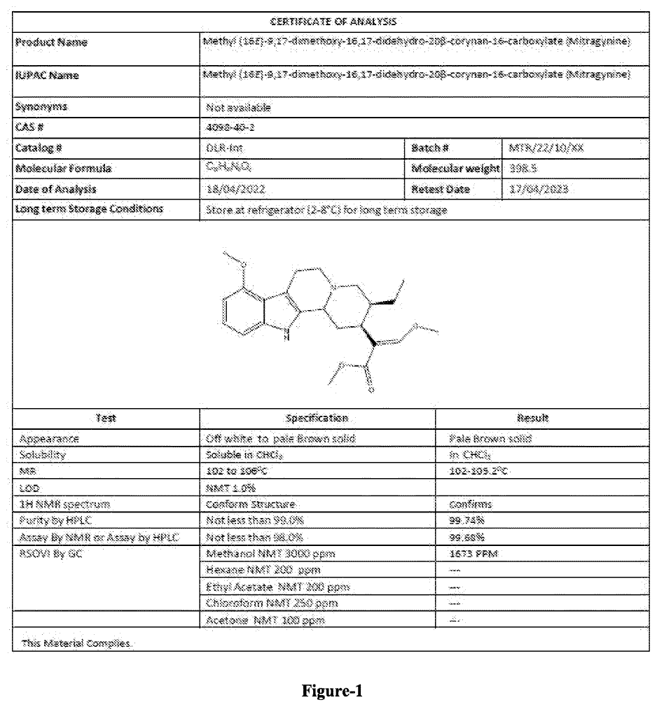

### DETAILED DESCRIPTION OF THE INVENTION

The following detailed description refers to the specific details and embodiments in which the invention may be practised. These embodiments are described in sufficient detail to enable those skilled in the art to practise the invention. Other embodiments may be utilized, and changes may be made without departing from the scope of the invention. The various embodiments are not necessarily mutually exclusive, as some embodiments can be combined with one or more other embodiments to form new embodiments.

Unless defined otherwise, all technical and scientific terms used herein have the same meaning as commonly understood by one of ordinary skill in the art to which the methods belong.

Further the embodiments described herein can be understood more readily by reference to the following detailed description, examples, and drawings. Methods described herein are merely illustrative of the principles of the present invention and are not limited to the specific embodiments presented in the detailed description, examples, and drawings. Numerous modifications and adaptations will be readily apparent to those of skill in the art without departing from the spirit and scope of the invention.

Where a range of values is provided, it is understood that each intervening value between the upper and lower limit of that range and any other stated or intervening value in that stated range, is encompassed within by the methods. The upper and lower limits of these smaller ranges may independently be included in the smaller ranges and are also encompassed within by the methods, subject to any specifically excluded limit in the stated range. Where the stated range includes one or both of the limits, ranges excluding either or both of those included limits are also included in the methods.

As stated above, Mitragynine has several pharmacological properties and are preferred with high purity and least possible impurities in pharmaceutical and medicinal preparations.

Accordingly, the present inventors have developed simple and commercially significant process for obtaining high purity Mitragynine for pharmaceutical and medicinal preparations.

The present invention discloses and describes an improved process for the production of high purity Mitragynine.

Said process involves purification of crude Mitragynine compound by chromatographic technique using mixture of non-polar aprotic solvent & polar aprotic solvent in presence of an organic base, wherein the process is performed in a silica column, comprising the steps by the process of the invention as described herein.

The present process for purification of Mitragynine comprises first loading the crude Mitragynine into silica column which is further eluted with mixture of non-polar aprotic and polar aprotic organic solvents in presence of organic base to obtain high purity Mitragynine compound.

The present purification process provides the purified Mitragynine compound with purity greater than 99% by HPLC.

In one aspect of the invention, there is provided a process for the production of high purity Mitragynine comprising the steps of:
(a) loading crude Mitragynine compound (1) in a silica column;
(b) eluting the column with mixture of non-polar aprotic and polar aprotic organic solvents in presence of an organic base;
(c) collecting the main fraction obtained in step (b) and concentrating under reduced pressure to obtain crude Mitragynine compound (2);
(d) loading the crude Mitragynine compound (2) obtained in step (c) into the silica column;
(e) eluting the column with mixture of non-polar aprotic and polar aprotic organic solvents;
(f) collecting the main fraction obtained in step (e) and concentrating under reduced pressure to obtain the material Mitragynine;
(g) dissolving the obtained material in step (f) in an alcohol to obtain alcoholic solution of Mitragynine and passing through the molecular sieves for multiple times; and
(h) concentrating the filtered alcoholic solution of Mitragynine obtained in step (g) under the reduced pressure to obtain high purity Mitragynine.

Accordingly, the crude Mitragynine is purified by the process provided by the present invention.

### Step (a):

In one embodiment of the present invention, the crude Mitragynine compound (1) employed in step (a) is 50-70% pure.

The crude Mitragynine compound used in the present invention may be prepared by the methods known in the state of art.

There are several methods known in the prior art for extracting and purifying Mitragynine. Some traditionally known and widely used methods for extraction of Mitragynine from plants includes maceration, percolation, reflux, Solvent Extraction, Soxhlet extraction. Amrianto et. al. (2021) discloses various methods reported in the art for extracting Mitragynine, both conventional and renewable technology that includes Solvent Extraction, Ultrasound assisted extraction (UAE), Accelerated solvent extraction (ASE) etc. followed by purification by acid-base techniques and chromatographic technique.

Some of these methods results in crude Mitragynine compound having purity 50-70%.

This crude Mitragynine compound having purity 50-70%, obtained by the above-mentioned known processes, is used in purification process as disclosed in the present invention. This crude compound can be purified to obtain high quality Mitragynine.

### Step (b):

In step (b) the column comprising crude Mitragynine compound (1) having 50-70% purity is eluted with mixture of non-polar aprotic organic solvent & polar aprotic organic solvent in presence of an organic base.

The non-polar aprotic organic solvent employed in step (b) may be selected from a group comprising of n-hexane, cyclohexane, pentane, heptane, carbon tetrachloride, benzene, toluene, or mixture thereof.

Preferably, the non-polar aprotic organic solvent employed in step (b) is n-hexane.

The polar aprotic organic solvent employed in step (b) may be selected from a group comprising of acetone, acetonitrile, dimethyl sulfoxide, dimethylformamide, tetrahydrofuran, dichloromethane, ethyl acetate, or mixture thereof.

Preferably the polar aprotic organic solvent employed in step (b) is ethyl acetate. The ratio (v/v) of non-polar aprotic organic solvent to polar aprotic organic solvent may vary from 80:20, 70:30, 60:40, 50:50, 40:60, 30:70, 20:80. Preferably, the ratio of non-polar aprotic organic solvent to polar aprotic organic solvent, as eluting solvents in step (b) is 60:40 (v/v).

The organic base employed in step (b) is selected from the group comprising of ammonia, amines, pyridine, aniline, or mixture thereof.

The organic base is preferably amine such as methylamine, dimethylamine, trimethylamine, ethylamine, diethylamine, benzylamine, butylamine, pentylamine, hexylamine, octylamine or the like.

More preferably, the organic base employed in step (b) is triethylamine.

The concentration of organic base used in step (b) ranges between 1% to 5% (v/v), preferably 2% (v/v).

In said step (b) the said column is eluted at a flow rate of 4-8 mL/min.

### Step (c):

In some embodiment of the invention, the main fractions obtained after elution in step (b) is collected by the process as known in the state of art.

In one preferred embodiment the collected solution of step (b) is concentrated under the reduced pressure using a rotary evaporator (Rotavapor).

### Step (d):

The crude Mitragynine compound (2) obtained in step (c) of the present process is loaded again to the silica column for further purification.

### Step (e):

In step (e) the column comprising crude Mitragynine compound (2) is eluted with mixture of non-polar aprotic solvent & polar aprotic solvent.

In one embodiment said step (e) is performed in absence of any organic base.

The non-polar aprotic organic solvent employed in steps (e) may be selected from a group comprising of n-hexane, cyclohexane, pentane, heptane, carbon tetrachloride, benzene, toluene or mixture thereof.

Preferably, the non-polar aprotic organic solvent employed in step (e) is n-hexane.

The polar aprotic organic solvent employed in step (e) may be selected from a group comprising of acetone, acetonitrile, dimethyl sulfoxide, dimethylformamide, tetrahydrofuran, dichloromethane, ethyl acetate, or mixture thereof.

Preferably the polar aprotic organic solvent employed in step (e) is ethyl acetate.

The ratio (v/v) of non-polar aprotic organic solvent to polar aprotic organic solvent may vary from 80:20, 70:30, 60:40, 50:50, 40:60, 30:70, 20:80. Preferably, the ratio of non-polar aprotic organic solvent to polar aprotic organic solvent, as eluting solvents in step (e) is 80:20 (v/v).

In said step (e) the said column is eluted at a flow rate of 5-10 mL/min.

### Step (f):

In some embodiment of the invention, the main fractions obtained after elution in step (e) is collected by the process as known in the state of art.

In one preferred embodiment the collected solution of step (e) is concentrated under reduced pressure using a rotary evaporator (Rotavapor).

### Step (g):

The material obtained in step (f) is dissolved in an alcohol to obtain alcoholic solution of Mitragynine and said solution is passed through the molecular sieves for multiple times.

The alcohol employed in step (g) is selected from the group comprising methanol, ethanol, n-propanol, isopropanol, n-butanol, and tert-butanol.

Accordingly, the alcoholic solution of Mitragynine obtained in this process incudes methanolic solution of Mitragynine, ethanolic solution of Mitragynine, n-propanolic solution of Mitragynine, iso-propanolic solution of Mitragynine, n-butanolic solution of Mitragynine, tert-butanolic solution of Mitragynine.

Said alcoholic solution of Mitragynine is passed through molecular sieves for multiple times, preferably two times, more preferably three times.

In one of the preferred embodiments, the obtained material in step (f) is dissolved in methanol to obtain methanolic solution of Mitragynine, which is passed through the molecular sieves for three times.

### Step (h):

The filtered alcoholic solution of Mitragynine obtained in step (g) is concentrated under the reduced pressure to obtain high purity Mitragynine.

In one preferred embodiment the filtered alcoholic solution of Mitragynine of step (g) is concentrated under the reduced pressure using a rotary evaporator (Rotavapor).

The present process is performed at room temperature.

In some embodiment of the invention there is provided a process for obtaining high purity Mitragynine, wherein said process comprises the steps of:
(a) loading crude Mitragynine compound (1) having purity 50-70% in a silica column;
(b) eluting the column with mixture of n-hexane and ethyl acetate in presence of triethylamine at a flow rate of 4-8 mL/min;
(c) collecting the main fraction obtained in step (b) and concentrating under reduced pressure to obtain crude Mitragynine compound (2);
(d) loading the crude Mitragynine compound (2) obtained in step (c) into the silica column;
(e) eluting the column with mixture of n-hexane and ethyl acetate at a flow rate of 5-10 mL/min;
(f) collecting the main fraction obtained in step (e) and concentrating under reduced pressure to obtain the material Mitragynine;
(g) dissolving the obtained material in step (f) in methanol to obtain methanolic solution of Mitragynine and passing through the molecular sieves for three times; and
(h) concentrating the methanolic solution of Mitragynine obtained in step (g) under the reduced pressure to obtain high purity Mitragynine.

In one embodiment the concentration under reduced pressure in step (c), (f) and (h) is performed using a rotary evaporator (Rotavapor).

The ratio (v/v) of n-hexane to ethyl acetate may vary from 80:20, 70:30, 60:40, 50:50, 40:60, 30:70, 20:80. Preferably, the ratio of n-hexane to ethyl acetate, as eluting solvents, in step (b) is 60:40 (v/v) and in step (e) is 80:20 (v/v).

The concentration of triethylamine in step (b) ranges between 1% to 5% (v/v), preferably 2% (v/v).

The process of the present invention provides improved yield of Mitragynine (30-50%) with purity greater than 99% by HPLC.

The obtained Mitragynine compound is free of any eluting solvent impurities such as n-hexane or ethyl acetate.

The obtained high purity Mitragynine compound is storage stable at 2° C.-8° C. for long term.

In some of the preferred embodiment of the present invention, the highly pure Mitragynine compound obtained according to present invention is suitable for the pharmaceutical preparations.

Certain specific aspect and embodiment of the present invention will be explained in detail with reference to the following examples, which are provided only for purposes of illustration and should not be construed as limiting the scope of the invention in any manner.

### EXAMPLES

### Crude Mitragynine Compound (Purity 50-70%):

The plant material for extraction of the Mitragynine extract was obtained from commercial vendors/suppliers in Indonesia and Thailand. The Mitragynine extract was obtained from the plant material by a process disclosed in Indian Patent Application No. IN202341010687 filed by the applicant in Indian Patent Office on Feb. 17, 2023, or Mitragynine extract may be obtained by any other process known in the art.

The crude Mitragynine compound (free base or in its salt form) having purity 50 to 70% may be obtained by the process disclosed in Indian patent application Nos. IN202341010930, IN202341010938 or IN202341010947, all three filed by the applicant in Indian Patent Office on Feb. 17, 2023, or any other process known in the art.

Mitragynine salt can be converted into its free base by any process known in the art.

### Example: Preparation of High Purity Mitragynine

The crude Mitragynine compound (free base) having purity 50-70% (1) was purified using column chromatography packed with silica gel.

The crude Mitragynine compound with purity around 50-70% (100 grams) was loaded into silica column.

The said column was eluted with the mixture of hexane and ethyl acetate at a ratio 60:40 in presence of 2% triethylamine as a mobile phase at a flow rate of 4-8 mL/min.

The main fraction obtained from the column after elution was collected and the solution was concentrated under reduced pressure using rotary evaporator (Rotavapor) to obtain crude Mitragynine compound (2) (45-65 grams).

The said crude Mitragynine compound (2) was again loaded into the silica column.

The said column was eluted with the mixture of hexane and ethyl acetate at a ratio 80:20 as a mobile phase at a flow rate of 5-10 mL/min.

The main fraction obtained from the column after elution is collected and the solution was concentrated under the reduced pressure using a rotary evaporator (Rotavapor) to obtain material (Mitragynine) (38-55 grams).

The obtained material comprising Mitragynine (38-55 grams) was dissolved in methanol (16-20 times of obtained material) to form methanolic solution of Mitragynine. Said methanolic solution is filtered by passing through molecular sieves for three times.

The filtered solution obtained above was concentrated under the reduced pressure using a rotary evaporator (Rotavapor) to obtain pale brown coloured Mitragynine solid compound.

All the steps were performed at room temperature.
Total time taken: 3 to 5 days.
Yield: 0.3 to 0.4 μm
% Yield: 30-50%

The obtained final compound was further subjected to analysis.

### Analytical Data:

Purity: 99.74% by HPLC.
An assay by NMR or by HPLC: 99.68%
n-hexane or ethyl acetate in final compound: Not detected.

The obtained pure Mitragynine compound is stable for long term storage, when stored at 2° C.-8° C.

1. A process for the production of high purity Mitragynine comprising the steps of:
(a) loading crude Mitragynine compound (1) in a silica column;
(b) eluting the column with mixture of non-polar aprotic and polar aprotic organic solvents in presence of an organic base;
(c) collecting the main fraction obtained in step (b) and concentrating under reduced pressure to obtain crude Mitragynine compound (2);
(d) loading the crude Mitragynine compound (2) obtained in step (c) into the silica column;
(e) eluting the column with mixture of non-polar aprotic and polar aprotic organic solvents;
(f) collecting the main fraction obtained in step (e) and concentrating under reduced pressure to obtain the material Mitragynine;
(g) dissolving the obtained material in step (f) in an alcohol to obtain alcoholic solution of Mitragynine and passing through the molecular sieves for multiple times; and
(h) concentrating the filtered alcoholic solution of Mitragynine obtained in step (g) under the reduced pressure to obtain high purity Mitragynine.

2. The process as claimed in claim 1, wherein the crude Mitragynine compound (1) employed in step (a) is 50-70% pure.

3. The process as claimed in claim 1, wherein the non-polar aprotic organic solvent and polar aprotic organic solvent in steps (b) and (e) are present at a ratio 80:20, 70:30, 60:40, 50:50, 40:60, 30:70, or 20:80.

4-5. (canceled)

6. The process as claimed in claim 1, wherein the non-polar aprotic organic solvent employed in steps (b) and (e) is selected from a group comprising of n-hexane, cyclohexane, pentane, heptane, carbon tetrachloride, benzene, toluene, or mixture thereof.

7. (canceled)

8. The process as claimed in claim 1, wherein the polar aprotic organic solvent employed in steps (b) and (e) is selected from a group comprising of acetone, acetonitrile, dimethyl sulfoxide, dimethylformamide, tetrahydrofuran, dichloromethane, ethyl acetate, or mixture thereof.

9. (canceled)

10. The process as claimed in claim 1, wherein the concentration of organic base employed in step (b) ranges between 1% to 5% (v/v).

11. The process as claimed in claim 1, wherein the concentration of organic base employed in step (b) is 2% (v/v).

12. The process as claimed in claim 1, wherein the organic base employed in step (b) is selected from the group comprising of ammonia, amines, pyridine, aniline, or mixture thereof.

13. The process as claimed in claim 12, wherein the organic base is amine selected from the group comprising of methylamine, dimethylamine, trimethylamine, ethylamine, diethylamine, benzylamine, butylamine, pentylamine, hexylamine, and octylamine.

14. (canceled)

15. The process as claimed in claim 1, wherein in step (b) the said column is eluted at a flow rate of 4-8 mL/min.

16. The process as claimed in claim 1, wherein in step (e) the said column is eluted at a flow rate of 5-10 mL/min.

17. The process as claimed in claim 1, wherein the concentration under reduced pressure in step (c), (f) and (h) is performed using a rotary evaporator.

18. The process as claimed in claim 1, wherein the alcohol employed in step (g) is selected from Methanol, Ethanol, n-Propanol, Isopropanol, n-Butanol and tert-Butanol.

19. (canceled)

20. The process as claimed in claim 1, wherein in step (g) the alcoholic solution of Mitragynine is passed through the molecular sieves for 2-3 times.

21. A process for the production of purified Mitragynine having above 99% HPLC purity comprising the steps of:
(a) loading crude Mitragynine compound (1) having purity 50-70% in a silica column;
(b) eluting the column with mixture of n-hexane and ethyl acetate in presence of triethylamine at a flow rate of 4-8 mL/min.;
(c) collecting the main fraction obtained in step (b) and concentrating under reduced pressure to obtain crude Mitragynine compound (2);
(d) loading the crude Mitragynine compound (2) obtained in step (c) into the silica column;
(e) eluting the column with mixture of n-hexane and ethyl acetate at a flow rate of 5-10 mL/min;
(f) collecting the main fraction obtained in step (e) and concentrating under reduced pressure to obtain the material Mitragynine;
(g) dissolving the obtained material in step (f) in methanol to obtain methanolic solution of Mitragynine and passing through the molecular sieves for three times; and
(h) concentrating the methanolic solution of Mitragynine obtained in step (g) under the reduced pressure to obtain high purity Mitragynine.

22. The process as claimed in claim 21, wherein the concentration under reduced pressure in steps (c), (f) and (h) is performed using a rotary evaporator.

23. The process as claimed in claim 21, wherein the ratio (v/v) of n-hexane to ethyl acetate is 80:20, 70:30, 60:40, 50:50, 40:60, 30:70, 20:80.

24. The process as claimed in claim 21, the ratio of n-hexane to ethyl acetate, in step (b) is 60:40 (v/v) and in step (e) is 80:20 (v/v).

25. The process as claimed in claim 21, wherein the concentration of triethylamine employed in step (b) ranges between 1% to 5% (v/v).

26. The process as claimed in claim 21, wherein the concentration of triethylamine in step (b) is 2% (v/v).

27. The process as claimed in claim 1, wherein the purity of obtained Mitragynine is above 99% by HPLC.

28. The process as claimed in claim 1, wherein the obtained Mitragynine compound is free of eluting solvent impurities.

29. (canceled)

30. The process as claimed in claim 1, wherein the obtained high purity Mitragynine compound is useful for preparation of pharmaceutical preparations and medicaments.

31. Purified Mitragynine having HPLC purity above 99%.
